(19) United States
(12) Patent Application Publication
(10) Pub. No.: US 2004/0031487 A1
(43) Pub. Date: Feb. 19, 2004

(54) Cover for kitchen

(75) Inventors: Burkhard Kruper, Menden (DE); Jozef Ninc, Filakovo (SK)

Correspondence Address: PEARNE & GORDON LLP, 1801 EAST 9TH STREET, SUITE 1200, CLEVELAND, OH, 44114-3108, US

(73) Assignee: Cramer SR s.r.o.

(21) Appl. No.: 10/642,940

(22) Filed: Aug. 18, 2003

(30) Foreign Application Priority Data
Aug. 18, 2002 (DE) 102 37 845.2

(51) Int. Cl. A61M 11/00

(52) U.S. Cl. 128/200.23

(57) ABSTRACT

Kitchen cover for kitchen furniture and like built-in members, in kitchenettes, living vans, campers, boat kitchens, hunting lodges, weekend houses and the like, as well as method for manufacturing a kitchen cover with a working plate 1 manufactured from a material curable in cold or hot, and comprising components like sinks, drip areas, cutting plates, rim strips and the like, wherein into said working plate (1) by impression into said material curable in cold or hot at least one component is integrated, in particular a burner insert 2 including a burner, which comprises means 7 for cooling said connecting area 3 between said curable material of said working plate 1 and said burner insert 2.

### BACKGROUND OF THE INVENTION

The present invention relates cover for use in kitchens as defined in the preamble of claim 1 and/or a method, respectively, for manufacturing a cover for kitchen as defined in the preamble of claim 10.

Covers for kitchen or working plates are widely used in present kitchens. The form a continuous border of built-in cupboards and since they avoid clefts and gaps they contribute to an improvement of hygienics in the kitchen area. In addition kitchen covers proved to be components easily to be assembled and permitting in simple manner to mount sinks and the like in a kitchen. Also in small kitchens in particular, like e.g. in living vans, campers, hunting lodges, weekend houses, boat kitchens and the like said easily mountable kitchen covers into which again in simple manner sinks and the like can be incorporated, due to the simple workability and the clean, gapless border have proved sound in kitchen furniture or built-in members.

In the traditional kitchen covers multiple and different materials are used, like e.g. stone plates, wood plates or also synthetic material plates. The essential point is that the plates in addition to an aesthetic surface in particular also are suited for the usual demands in a kitchen. Thus, the working plates e.g. have to be temperature-proof and scratchproof. A provision for this is a given hardness of the kitchen cover.

A comparatively novel material for forming kitchen covers is glass fiber reinforced plastics (GFRP) which in addition can have a coating (gel coat) which simultaneously is hard and represents a decorative coating. In these kitchen covers made from GFRP it also is known to form into the not yet cured GFRP material components of kitchen covers, like e.g. sinks, scrap sinks, drain, rim strips and the like, so that after curing of the kitchen cover an integrated member with the corresponding components is available.

This provides the advantage that said kitchen cover can already be preassembled completely so that during installation of the kitchen only the preassembled kitchen cover has to be mounted and additional time-consuming work for arranging the components in the kitchen cover, like e.g. sinks etc., can be done without. A drawback of this technology, however, lies in that not all components usually provided for in a kitchen cover, can be formed into the kitchen cover. Thus, it is not possible to from cooking depressions in kitchen covers made from GFRP since cooking depressions during operation are temperature-stressed so that the adjacent GFRP material might be damaged. Correspondingly, cooking depressions still have to be separately mounted in corresponding patches of said kitchen cover, wherein then expensive sealing and fixation materials are used between kitchen cover and cooking depressions. This, however, results in the essential disadvantage that the kitchen cover no longer represents a completely premanufactured component but again additional installation work has to be carried out.

### SUMMARY OF THE INVENTION

It is, therefore, the object of the present invention to create a kitchen cover which as being a completely integrated component is to also include at least a cooking depression. In addition the kitchen cover is to be manufacturable easily and economically and to answer the demands with respect to hardness and decorative appearance.

This object is solved by a kitchen cover showing the features of claim 1 as well as a method for manufacturing a kitchen cover with the features of claim 10. Preferred embodiments are subject of the depending claims.

The essential aspect of the present invention lies in that components of the kitchen cover like sinks, drip areas, cutting plates and the like and also a part of a burner in particular, e.g. of an electric or gas burner, namely a so-called burner insert are integrated into said working plate, namely by impression into a material curable by heat or cold. However, to avoid that the working plate is damaged by temperature load of the burner insert in the connecting area between working plate material and burner insert during operation of the burner, further means for cooling the connecting area are provided for.

In this way it is possible to create a completely integrated kitchen cover in which after assembly of the kitchen cover also the burner insert is integrated. Preferably, the burner insert is embodied such that it represents a part of a common burner, in particular a common burner preferably forms the central portion of the burner insert.

The means for cooling the connecting area between the curable material of the working plate and the burner insert can include all common cooling means, like e.g. water cooling, air cooling etc., wherein cooling of the connecting area also includes that adjacent areas, on the sides of said burner insert in particular, are cooled, i.e. indirect cooling is present.

In particular, it turned out to be advantages to provide for a spacing area as cooling means, which is part of the burner insert or is formed by the burner insert, such that the burner is spaced from the connecting area of the working plate to such extent that due to the cooling by ambient air the temperature in the connecting area is kept so low that no damage occurs on the curable material of the working plate. Depending on the kind of burner, the spacing area can be dimensioned differently. The spacing area only has to be dimensioned such that the occurring heat of the burner over the distance between burner and connecting area between working plate and burner insert results in a temperature in the connecting area, which is inoffensive for the working plate material. Correspondingly, the burner insert can have a codification which depending on the chosen spacing area only permits burners with a given output so that damage of the working plate material is avoided.

Preferably the spacing area of the burner insert is embodied as diskshaped, in particular circular metal sheet in the center whereof a burner insert receiving opening is provided for. The spacing area in this simple embodiment is formed by that the disk diameter is chosen correspondingly large so that for the heat transfer from the burner up to the connecting area between burner insert and working plate a sufficiently long path is available, which has the effect that the temperature in the connecting area is sufficiently low. By the heat transfer path chosen sufficiently long, sufficient cooling by ambient air is guaranteed. Herein, it also turned out to be sensible to minimize heat transfer by perforations and slits at the rim of the burner insert. As further measurement a material with low thermal conductivity can be chosen for the spacing area, also this will contribute to the temperature in the connecting area being kept low.

Beside a one-piece embodiment of spacing area and burner insert, of course, also the burner insert and the spacing area can be formed as separate components, wherein if required also the burner insert per see and the spacing area per see again can be realized in several parts.

As materials for the burner insert and/or the spacing area metallic materials, enameled metals in particular, turned out to be favorable. On the other hand, the working plate is manufactured from a material curable by heat or cold, a synthetic material in particular. Here, preferably, materials chosen from the group consisting of glass fiber reinforced plastics (GFRP), glass fiber reinforced polyester in particular, as well as SMC materials (sheet molding compounds), including carbon fiber reinforced plastics, are used. It is important that the materials for the spacing area and/or burner insert are poor thermal conductors.

Preferably in the area of the burner insert, the kitchen cover comprises a depression so that overbooking water or the like can be caught. The recess can therein either partly be formed by the working plate or by the burner insert. Correspondingly, the vertical side walls or parts of the depression bottom also are formed from the material of the working plate or from the material of the burner insert. At least in the bottom area of the depression, however, the burner insert is arranged, wherein the bottom area preferably again comprises a section shaped like the frustum of a pyramid so that the burner per se and the cooking stuff arranged thereabove is elevated from the surrounding depression bottom. The frusto-pyramidal part of the depression bottom again can completely or partly be formed either by the working plate or by the burner insert.

In the connecting area it is advantageous, if the working plate ends in flush with the burner insert for avoiding clefts and gaps in which dirt can collect. The flush end can be achieved as well if the burner insert engages with the working plate from top and also is pressed into the material of the working plate from the front face.

To guarantee anchoring of the burner insert in the working plate, it is preferred to realize the marginal area of the burner insert at a bend or an angle. Such an embodiment can also serve for generating the flush ending between working plate and burner insert.

### BRIEF DESCRIPTION OF DRAWINGS

Further embodiments, characteristics and features of the present invention will now become evident from the following detailed description of preferred embodiments with reference to the attached drawing. The drawings therein in purely schematic manner show the following.

FIGS. 1 to 6 show partly sectional views of a kitchen cover in the area of a cooking depression with integrated insertion of a burner insert into a working plate;

FIGS. 7 to 9 show partly top views onto a kitchen cover in the area of a cooking depression with a different number of burner inserts and

FIG. 10 in partial figures (a to c) partly shows top views onto kitchen covers in the area of cooking depressions with different shapes of the burner inserts.

### DETAILED DESCRIPTION OF PREFERRED EMBODIMENT

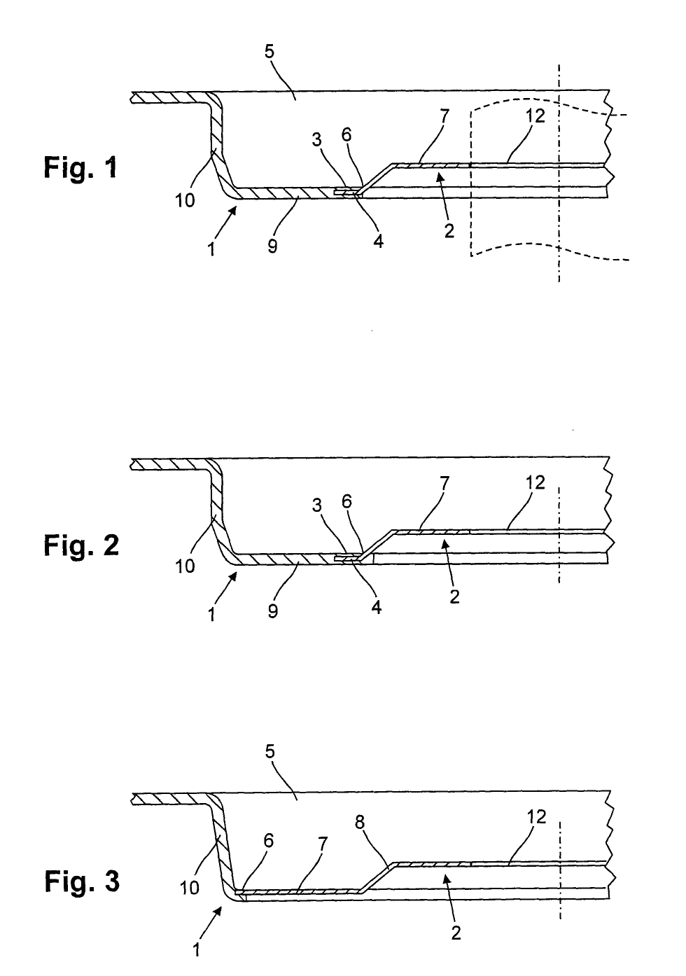

FIG. 1 shows a first embodiment of the kitchen cover with burner insert 2 integrated in accordance with the present invention, in he area of a cooking depression 5. Said cooking depression 5 in the embodiment is formed into the working plate 1, wherein said working plate 1 comprises a vertical working plate area 10 and a horizontally extending platform bottom 9. Into said platform bottom 9 said burner insert 2 is impressed, wherein said burner insert 2 has an in total frusto-pyramidal shape. In the marginal area said burner insert 2 comprises a bend 4 which in connection with the impression in the connection area 3 is formed such that said bottom section 9 of said working plate 1 at 6 ends in flush with said burner insert 2. Said burner insert 2 further is embodied such that over the spacing area 7 it has a sufficiently large distance to the burner insert opening 12 to said connecting area 3 with said working plate 1, so that at a temperature development in the area of said burner reception opening 12 no damaging effect is caused to the material of said working plate 1.

In principle similar embodiments of inclusion of a burner insert 2 into said working plate 1 are shown in the following FIGS. 2 to 6 correspondingly, in the following only the differing aspects are dealt with.

In FIG. 2 the embodiment only differs in that the marginal area of said burner insert 2 is not inserted and impressed through the front face of said bottom area 9 of said working plate 1 into the material of said working plate 1, but from the upper side of said working plate 1. This means that said working plate 1 still a little bit extends in direction of said burner insert opening 12 and grips under said burner insert 2.

The embodiment of FIG. 3 differs from the preceding embodiments essentially in that said working plate 1 essentially does no longer comprise horizontal sections in said bottom area 9 but only the vertical section 10. In the end of said vertical section 10 then again said marginal area of said burner insert 2 is laterally inserted into the material of said working plate 1, wherein said marginal area of said burner insert 2 does no longer show any chamfering or bending. Nevertheless, the entire shape of the embodiment of depression is essentially the same, since said burner insert 2 includes a frusto-pyramidal section 8.

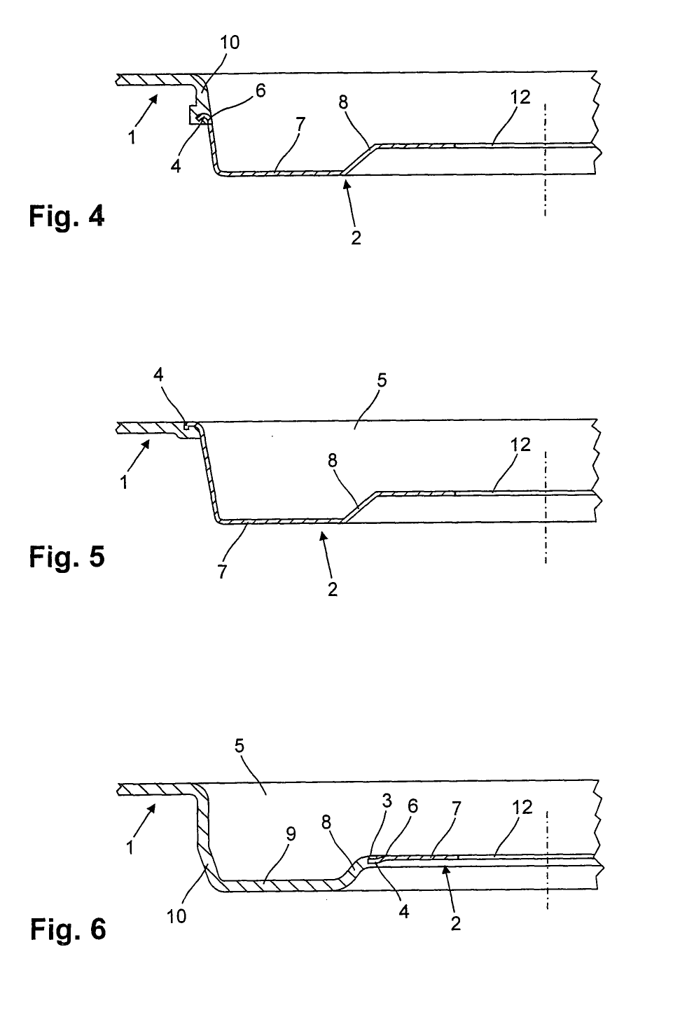

In the embodiment under FIG. 4 not only the horizontal depression bottom area 9 is substituted from by a portion of said burner insert but in addition also a portion of said vertical section 10 so that said burner insert 2 in cross-section takes a depression shape itself.

A consequent further development of this conception is shown in FIG. 5 where it is shown that said working plate 1 essentially does not longer include a depression section at all, but that said cooking depression 5 completely is formed by said burner insert 2. Here, too, said marginal section bent at 4, of said burner insert 2 engages with said working plate 1 from top.

In FIG. 6 again an embodiment is shown which rather corresponds to the embodiments of FIGS. 1 and 2, since said working plate 1 here again comprises a depression section 5 with a vertical section 10 and a horizontal depression area 9. Additionally, however, also said frusto-pyramidal section 8 at least partly is formed by said working plate 1 so that said burner insert 2 essentially can be formed as flat disk. Only in the marginal area in which said burner insert 2 engages and is pressed with the material of said working plate 2, said burner insert 2 has a slight bend in order to guarantee a flush transition from said working plate 1 to said burner insert 2 on said surface at 6. Said flush and almost smooth transition from said working plate 1 to said burner insert 2 in said connecting area 3 permits a clean, since gapless transition and thus severely increases the hygienic conditions in said cooking depression 5.

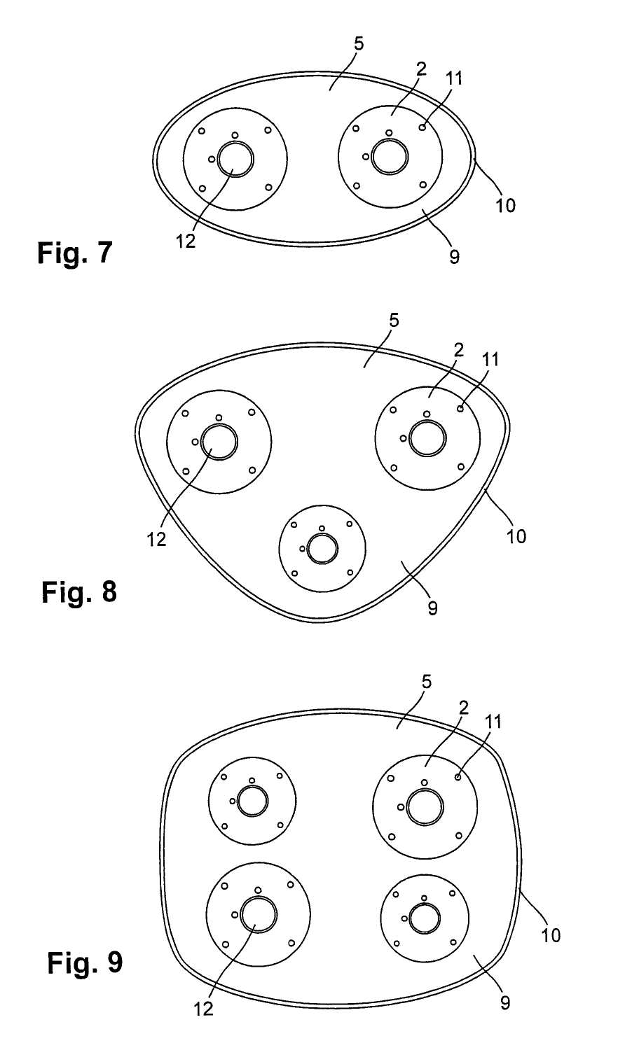

FIGS. 7 to 9 show different cooking depressions 5 with different numbers of burner inserts 2. In FIG. 7 a twin-flame cooking depression 5 is shown, whereas in FIG. 8 a three-flame cooking depression 5 can be seen. FIG. 9 again shows a four-flame platform 5. In addition, the embodiments of FIGS. 7 to 9 differ in that burner inserts 2 of different sizes are provided for. This corresponds to the different burner outputs of the different burners provided for said burner inserts. Said burner inserts in addition comprise grate receptions 11 in form of depressions in which corresponding grates can be inserted.

In addition the embodiments of FIGS. 7 to 9 differ in the different shapes of said cooking depression 5 which can assume different shapes like e.g. oval, almost triangular, square or four-cornered. Of course, also all other shapes are conceivable and possible.

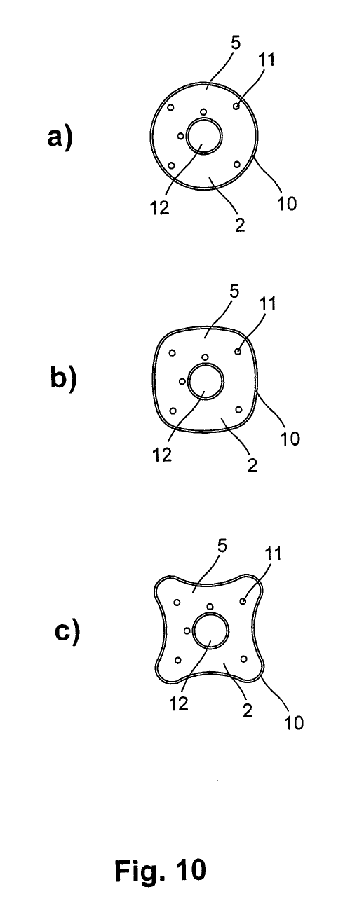

An example for this is given in FIG. 10 in partial views (a to c) which show single-flame cooking depressions 5 with burner inserts 2 with also differentiating outer contours. Said cooking depressions 5 of FIG. 10 further differ from said cooking depressions of FIGS. 7 to 9 in that said working plate 1 comprises only one vertical section 10 whereas said horizontal depression bottom area is exclusively formed by said burner insert 2, as e.g. is the case also in the embodiment of FIG. 3. Corresponding ones of said embodiments, of course, also are possible for burner inserts 2 with depression-shaped cross-section corresponding to the embodiments of FIGS. 4 and 5.

What is claimed

1. A kitchen cover for kitchen furniture and like built-in members, in kitchenettes, living vans, campers, boat kitchens, hunting lodges, weekend houses and the like, with a working plate (1) manufactured from a material curable in cold or hot, and comprising components like sinks, drip areas, cutting plates, rim strips and the like,
WHEREIN at least one of said components is integrated into said working plate (1) by impression into said material curable in cold or hot.

2. The kitchen cover as defined in claim 1,
WHEREIN said working plate comprises at least one burner insert (2) including a burner, which comprises means (7) for cooling said connecting area (3) between said curable material of said working plate (1) and said burner insert (2).

3. The kitchen cover as defined in claims 1 or 2,
WHEREIN said means (7) for cooling include a spacing area cooled by ambient air and dimensioned such that during operation of said burner in the temperature gradient resulting over said spacing area (7), in said connecting area (3) between said curable material of said working plate (1) and said burner insert (2) a temperature agreeable for said curable material will result.

4. The kitchen cover as defined in claim 3,
WHEREIN said spacing area (7) is part of said burner insert (2).

5. The kitchen cover as defined in one of the preceding claims,
WHEREIN said burner insert (2) and/or said spacing areas (7) are multi-part ones.

6. The kitchen cover as defined in one of the preceding claims,
WHEREIN said burner insert (2) and/or said spacing area (7) are formed from one metallic material, an enameled metal in particular.

7. The kitchen cover as defined in one of the preceding claims,
WHEREIN said burner includes a gasburner and/or electroburner in particular.

8. The kitchen cover as defined in one of the preceding claims,
WHEREIN said working plate (1) includes at least one material of the group consisting of GFRP (glass fiber reinforced plastics), glass fiber reinforced polyester in particular and SMC (sheet molding compounds) incl. carbon fiber reinforced plastics.

9. The kitchen cover as defined in one of the preceding claims,
WHEREIN said kitchen cover in the area of said burner insert (2) comprises a depression (5), wherein said burner insert at least partly forms the bottom of said depression or itself is depression-shaped, and wherein said bottom of said depression in particular at least partly is embodied in frusto-pyramidal shape.

10. The kitchen cover as defined in one of the preceding claims,
WHEREIN said burner insert (2) in said connecting area (3) between burner insert (2) and working plate (1) ends in flush with the surface of said working plate (1), wherein said burner insert (2) either engages into the upper surface of said working plate (1) and/or in particular in case of central engagement seen in cross-section, in the front face area of said working plate (1) is embodied in bent or angular form.

11. A method for manufacturing a kitchen cover as defined in one of the preceding claims in particular,
WHEREIN a working plate (1) is manufactured from a material curable in cold or heat, wherein in at least one recess of said working plate (1) a component, like a sink, cutting board, a burner insert (2) or the like is impressed with the marginal area thereof into the curable material in the marginal area of said recess of said working plate (1) and in which subsequently the curable material is cured so that said working plate (1) includes the component integrally.
